(19) United States
(12) Patent Application Publication
(10) Pub. No.: US 2021/0363494 A1
(43) Pub. Date: Nov. 25, 2021

(54) Universal Human Induced Pluripotent Stem Cells And Method Of Forming The Same

(71) Applicant: National Central University, Taoyuan City (TW)

(72) Inventors: Akon Higuchi, Taoyuan City (TW); Tzu-Cheng Sung, Kaohsiung City (TW)

(73) Assignee: National Central University, Taoyuan City (TW)

(21) Appl. No.: 16/939,871

(22) Filed: Jul. 27, 2020

(30) Foreign Application Priority Data
May 20, 2020 (TW) 109116761

(51) Int. Cl. C12N 5/074; C12N 5/0735; C12N 15/85; C07K 14/74

(52) U.S. Cl. CPC C12N 5/0696; C12N 5/0606; C12N 5/0607; C12N 15/85; C07K 14/70539; C12N 2501/602; C12N 2510/00; C12N 2501/604; C12N 2501/605; C12N 2501/606; C12N 2501/608; C12N 2502/45; C12N 2501/603

(57) ABSTRACT

Universal human induced pluripotent stem cells (universal hiPSCs) and a method of forming the same are provided in the disclosure, including the following steps: providing a first cell group including human stem cells; providing a second cell group including human mononuclear cells; in some embodiments, the second cell group further includes human stem cells, in which the human stem cells of the second cell group are allogenic cells from the first cell group; mixing the first cell group and the second cell group to form cell mixture; maintaining the cell mixture at a temperature below 30° C. for at least one day; reprogramming the human stem cells of the cell mixture to obtain universal hiPSCs. The universal hiPSCs includes human leukocyte antigen-1 (HLA class I) gene and human leukocyte antigen-2 (HLA class II) gene, but no HLA class I and HLA class II expressions.

### CROSS-REFERENCE TO RELATED APPLICATION

This application claims priority to Taiwan Application Serial Number 109116761, filed May 20, 2020, which is herein incorporated by reference in its entirety.

### BACKGROUND

### Field of Invention

This present disclosure relates to human induced pluripotent stem cells (hiPSCs or hiPSC) and a method of forming the same; in particular, the present disclosure relates to hiPSCs showing no expression of human leukocyte antigen-1 (HLA-1 or HLA Class I) and human leukocyte antigen-2 (HLA-2 or HLA Class II) and a method of forming the same.

### Description of Related Art

Human pluripotent stem cells (hPSCs or hPSC) are cells with the potential to be differentiated into any cell types, including human embryonic stem cells (hESCs) or human induced pluripotent stem cells (hiPSCs) which are formed by reprogramming somatic cells back into undifferentiated state, which play important roles in cell therapy. However, regardless of hESCs or hiPSCs, unique expression forms of HLA class I and HLA class II are preformed depending on the difference of the source subjects. The difference of expression form of HLA class I and HLA class II in different subjects results in recognition of hESCs or hiPSCs by the recipients' immune systems, induction of immune responses, or even life-threatening risks to the recipients' lives while hESCs or hiPSCs are transplanted into other subjects (allogeneic transplantation).

The current method for improving the immune response during allogeneic transplantation of hPSC is primarily directed to gene editing (such as clustered regularly interspaced short palindromic repeat; CRISPR), which deletes the gene fragments of HLA class I or HLA class II in hESCs or hiPSCs to avoid the immune response while allogeneic transplantation of hESCs or hiPSCs. However, the gene editing technology is not only complicating to be operated, but it also remains high concerns about the security of hESCs or hiPSCs processed by gene editing for the reasons that at the current stage, there are still many unknowns about the regulatory mechanism in the cells, and the side effects are unclear.

Therefore, how to improve the current method of forming hESCs or hiPSCs without changing the chromosomal genes and to obtain hiPSCs that induce no immune response during allogeneic transplantation is a problem to be solved.

### SUMMARY

A method of forming universal human induced pluripotent stem cells is provided in some embodiments of the present disclosure, including the following steps: providing a first cell group including human stem cells; providing a second cell group including human mononuclear cells, in which an expression form of human leukocyte antigen-1 (HLA class I) or an expression form of human leukocyte antigen-2 (HLA class II) of the human mononuclear cells are different from an expression form of HLA class I or an expression form of HLA class II of the first cell group; mixing the first cell group and the second cell group to form a cell mixture; maintaining the cell mixture below 30° C. for at least one day; and reprogramming the human stem cells in the cell mixture, and obtaining universal human induced pluripotent stem cells, in which the universal human induced pluripotent stem cells include HLA class I gene and HLA class II gene, but no HLA class I and the HLA class II expressions.

In some embodiments, at a step of providing the first cell group including human stem cells, the first cell group is obtained from a first amniotic fluid, dental pulp, umbilical cord, umbilical cord blood, adipose, marrow, or combination thereof.

In some embodiments, at a step of providing the second cell group including the human mononuclear cells, the second cell group is obtained from spinal fluid, second amniotic fluid, blood, or combination thereof.

In some embodiments, at the step of providing the first cell group including human stem cells, in which the first cell group is obtained from first amniotic fluid, and at the step of providing the second cell group including the human mononuclear cells, in which the second cell group is obtained from second amniotic fluid, the first amniotic fluid and the second amniotic fluid are obtained from different subjects.

In Some Embodiments, the First Amniotic Fluid and the Second Amniotic Fluid are at Least 0.5 mL, Respectively.

In some embodiments, at the step of providing the second cell group including the human mononuclear cells, the second amniotic fluid includes a plurality of amniotic fluids derived from different subjects, in which the plurality of amniotic fluids include a plurality of human mononuclear cells.

In some embodiments, at the step of providing the first cell group including human stem cells, the first cell group is obtained from the first amniotic fluid, and at the step of providing the second cell group including the human mononuclear cells, the second cell group is human mononuclear cells.

In some embodiments, the step of mixing the first cell group and the second cell group includes mixing the first amniotic fluid and a buffer including the human mononuclear cells to form the cell mixture, in which the first amniotic fluid is at least 0.5 mL, and a concentration of the human mononuclear cells in a cell culture dish containing the cell mixture is 1.5×10⁴ to 1.5×10⁶ cells/mL.

In some embodiments, the step of reprogramming the human stem cells in the cell mixture includes transfecting a vector containing nucleic acids of nucleic reprogramming factors into the human stem cells in the cell mixture.

In some embodiments of the present disclosure, a universal human induced pluripotent stem cell is provided, including HLA class I gene and HLA class II gene, but no expression of HLA class I and HLA class II.

In some embodiments of the present disclosure, a differentiated cell is provided, including HLA class I gene and HLA class II gene, but no HLA class I and HLA class II expressions.

Universal hiPSCs and the method of forming the same are provided in the present disclosure, which are not only simply operated, but the formed universal hiPSCs are highly safe because the universal hiPSCs are not processed by gene editing. In addition, they are applicable in allogeneic transplantation and have high application value in clinical uses.

### BRIEF DESCRIPTION OF THE DRAWINGS

The invention can be more fully understood by reading the following detailed description of the embodiment, with reference made to the accompanying drawings as follows:

FIG. 1 depicts the diagrams of a method of forming universal hiPSCs in some embodiments of the present disclosure;

FIGS. 2A to 2C depict the images of cell characteristic analysis of hiPSC (mix-5) in Example 2 of the present disclosure; FIG. 2A is images of immunostaining of the pluripotent proteins in hiPSC (mix-5), in which the blue in (i)-(iv) is the nucleus, the green in (i) is Oct4, the green in (ii) is Sox2, the red in (iii) is Nanog, the red in (iv) is SSEA-4; FIG. 2B is images of embryoid bodies derived from hiPSC (mix-5) and immunostaining images of marker proteins in each embryo layer of the embryoid bodies, in which (i) is the cell types of the embryoid bodies differentiated from hiPSC (mix-5); in addition, the blue in (ii) is nuclear staining, and the green in (ii) is SMA, the red in (iii) is GFAP, and the green in (iv) is AFP; FIG. 2C depicts images of teratomas and immunohistologic sections of teratomas after hiPSC (mix-5) is subcutaneously injected into mice, in which (i) is mice appearance while the teratoma is formed, and (ii)-(iv) are immunohistologic sections of teratomas, representing the tissue morphology of the endoderm (ii), mesoderm (iii) and ectoderm (iv) of the embryoid bodies, respectively;

FIG. 3A depicts microscope images of hiPSC (mix-2) and hiPSC (mix-5) at each culture time point after differentiation treatment, in which (i)-(iv) are the differentiation process of hiPSC (mix-2), and (v)-(viii) are the differentiation process of hiPSC (mix-5) in Example 3 of the present disclosure;

FIG. 3B depicts immunostaining images of detecting specific proteins in cardiomyocytes differentiated from hiPSC (mix-5) in Example 3 of the present disclosure;

FIGS. 4A to 4D depict the figures of the expression ratio of HLA class I and HLA class II in each cell group analyzed by the flow cytometry in Example 4 of the present disclosure, in which the cell groups include cardiomyocytes derived (differentiated) from hESC (H9), cardiomyocytes derived from hiPSC0077, cells related to hiPSC (single) which including cells before isolation to cells after differentiation, cells related to hiPSC (mix-2) which including the cells before isolation to the cells after differentiation, and cells related to hiPSC (mix-5) which including the cells before isolation to the cells after differentiation; and

FIGS. 5A to 5C depict cell survival ratio (FIG. 5A), concentration of cytokine IFN-γ (FIG. 5B), and concentration of cytokine IL-6 (FIG. 5C) of each cell group (hESC (H9), hiPSC0077, hiPSC (single), hiPSC (mix-2), hiPSC (mix-5)) before and after mononuclear cells (MN) treatment in Example 5 of the present disclosure.

### DETAILED DESCRIPTION

The following disclosure provides many different embodiments, or examples, for implementing different features of the invention. Specific examples of components and arrangements are described below to simplify the present disclosure. These are, of course, merely examples and are not intended to be limiting. The following disclosure provides detailed description of many different embodiments, or examples, for implementing different features of the provided subject matter. In the following description, many specific details are set forth to provide a more thorough understanding of the present disclosure. It will be apparent, however, to those skilled in the art, that the present disclosure may be practiced without these specific details.

As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

Although a series of operations or steps are used below to describe the method disclosed herein, an order of these operations or steps should not be construed as a limitation to the present invention. For example, some operations or steps may be performed in a different order and/or other steps may be performed at the same time. In addition, all shown operations, steps and/or features are not required to be executed to implement an embodiment of the present invention. In addition, each operation or step described herein may include a plurality of sub-steps or actions.

As used herein, the term “nucleic reprogramming factors” indicates any protein factors or protein groups that can induce somatic cells to human induced pluripotent stem cells.

HLA class I gene and HLA class II gene are a series of closely linked genetic locus, which preforms high degree of polymorphism. The expression forms of the encoded HLA class I and HLA class II are different along with different subjects. Therefore, HLA class I and HLA class II serve as the markers for immune cells to distinguish between themselves and allogeneic cells. As used herein, “universal human induced pluripotent stem cells” (universal hiPSCs) represent as hiPSCs that do not express human HLA class I and human HLA class II, in which universal means that it can be applied to different subjects.

As used herein, “differentiated cell” means the cell which is specialized to have special shapes and/or physiological functions. For example, hiPSCs can be differentiated (derived) into differentiated cells according to the differentiation method of the present disclosure, in which differentiated cells can include, for example, incompletely differentiated precursor cells and fully differentiated somatic cells.

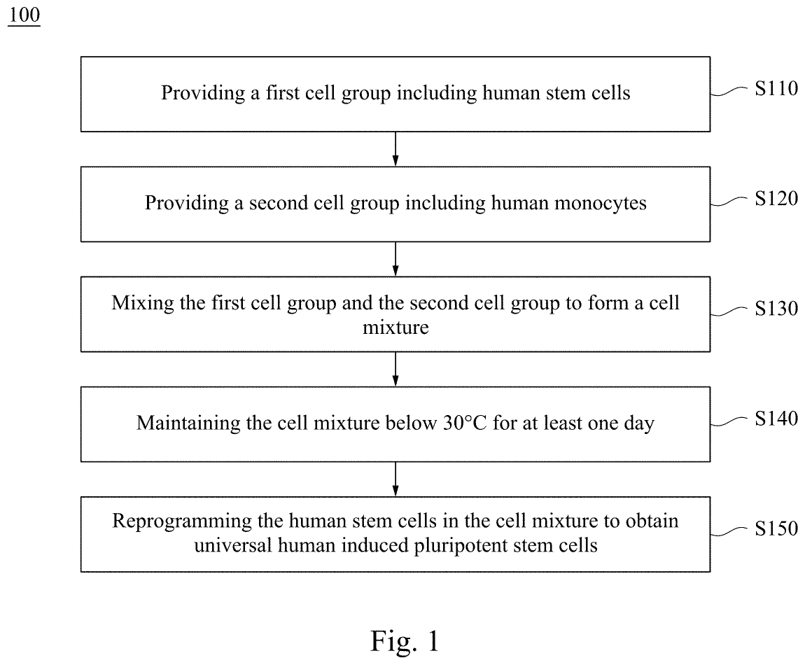

Please refer to FIG. 1. A method of forming universal human induced pluripotent stem cells is provided in the present disclosure, including the following steps: step S110, providing a first cell group including human stem cells; step S120, providing a second cell group including human mononuclear cells, in which an expression form of HLA class I and an expression form of HLA class II of the human mononuclear cells are different from an expression form of HLA class I and an expression form of HLA class II of the first cell group; step S130, mixing the first cell group and the second cell group to form a cell mixture; step 140, maintaining the cell mixture below 30° C. for at least one day; and step S150, reprogramming the human stem cells in the cell mixture and obtaining universal human induced pluripotent stem cells, in which the universal human induced pluripotent stem cells include HLA class I gene and HLA class II gene, but no HLA class I and HLA class II expressions. In some embodiments, human stem cells and human mononuclear cells are obtained from different subjects, respectively, and no genetic relationship between the subjects for ensuring the expression forms of HLA class I or the expression forms of HLA class II of the human mononuclear cells are different from which of the first cell group.

That is, it is firstly discovered that the human stem cells that do not express HLA class I and HLA class II are contained in human tissue in some embodiments of the present disclosure. Since human mononuclear cells are capable of removing the human stem cells with different HLA class I expression forms or different HLA class II expression forms, human mononuclear cells remove the human stem cells that express HLA class I and HLA class II in the first cell group and leave the human stem cells that do not express HLA class I and HLA class II, and the ratio of the human stem cells that express no HLA class I and HLA class II is increased according to the characteristics that the expression forms of HLA class I and the expression forms of HLA class II of the first cell group are different from which of the human mononuclear cells. Furthermore, the human stem cells processed by the human mononuclear cells in the first cell group are reprogrammed, and universal hiPSCs that do not express HLA class I and HLA class II are obtained.

In some embodiments, at the step of providing the first cell group including human stem cells, the first cell group is obtained from the first amniotic fluid (including amniotic fluid stem cells; AFSCs), dental pulp (including dental pulp stem cells), umbilical cord (including umbilical cord-derived stem cells), umbilical cord blood (including umbilical cord blood-derived stem cells), adipose (including adipose-derived stem cells), marrow (including marrow stem cells) or combination thereof. In some embodiments, the step of providing the first cell group including the human stem cells further includes providing somatic cells, and then selecting the human stem cells from the somatic cells. In one embodiment, according to the low activation degree of house-keeping genes in the stem cells, the human stem cells are obtained from the somatic cells by screening the somatic cells, including, for example, screening the somatic cells with low activation degree of housekeeping genes, thereby obtaining the human stem cells. In another embodiment, the stem cells are obtained from the somatic cells, including screening the cells with specific proteins of the stem cells, thereby obtaining the human stem cells.

In some embodiments, at the step of providing the second cell group including the human mononuclear cells, the second cell group is obtained from body fluid or blood including human mononuclear cells, for example, spinal fluid, second amniotic fluid, blood, or combination thereof.

In some embodiments, at the step of providing the first cell group including human stem cells, in which the first cell group is obtained from the first amniotic fluid, and at the step of providing the second cell group including the human mononuclear cells, in which the second cell group is obtained from the second amniotic fluid, the first amniotic fluid and the second amniotic fluid are obtained from different subjects. In one embodiment, the first amniotic fluid and the second amniotic fluid are at least 0.5 mL, respectively. This volume of the amniotic fluid can ensure the sufficient AFSCs are maintained after AFSCs in the first amniotic fluid are treated with the human mononuclear cells in the second amniotic fluid; however, the selected AFSCs are not enough if the volume of the amniotic fluid is lower than 0.5 mL. In one embodiment, the second cell group including human mononuclear cells further includes a plurality of amniotic fluids from different subjects, in which AFSCs in the first amniotic fluid are obtained by a plurality of human mononuclear cells from the plurality of amniotic fluids. In general, the more amniotic fluids as the second cell group people provides (i.e., more kinds of human mononuclear cells with different HLA class I or HLA class II), the better screening efficiency of AFSCs that do not express HLA class I and HLA class II the screening performs.

In another embodiment, at the step of providing the first cell group including human stem cells, the first cell group is obtained from the first amniotic fluid, and at the step of providing the second cell group including the human mononuclear cells, the second cell group is human mononuclear cells. In one embodiments, the step of mixing the first cell group and the second cell group includes mixing the first amniotic fluid and the human mononuclear cells to form the cell mixture, in which the first amniotic fluid is at least 0.5 mL, and the concentration of the human mononuclear cells in the cell culture dish containing the cell mixture is 1.5×10⁴ to 1.5×10⁶ cells/mL, for example, 1.5×10⁴ cells/mL, 1.5×10⁵ cells/mL, 1.5×10⁶ cells/mL, or a concentration in any aforementioned intervals. It should be emphasized that it will be difficult to obtain universal hiPSCs if the functional concentration of human mononuclear cells is too low or too high. For example, the screening efficiency of AFSCs that does not express HLA class I and HLA class II is poor if the function concentration of human mononuclear cells is too low; thus, it is difficult to obtain universal hiPSCs. Conversely, AFSCs that does not express HLA class I and HLA class II are removed by mononuclear cells if the function concentration of human mononuclear cells is too high; thus, it is also difficult to obtain universal hiPSCs.

In some embodiments, at the step of maintaining the cell mixture below 30° C. for at least one day, the temperature below 30° C. includes 1° C., 2° C., 3° C., 4° C., 5° C., 6° C., 7° C., 8° C., 9° C., 10° C., 15° C., 20° C., 25° C., 30° C. or any aforementioned values, and at least 1 day includes one day, two days, more than three days, the like, or any appropriate time that keeps the human stem cells in the cell mixture alive.

In some embodiments, after the step of maintaining the cell mixture is maintained below 30° C. for at least one day, centrifuge the cell mixture and obtain supernatant and precipitate, in which the precipitate contains human stem cells. In one embodiment, the step of centrifuging the cell mixture includes centrifuging the cell mixture at 300 xg to 400 xg, and the time for centrifugation lasts for 1 minute, 2 minutes, 3 minutes, 4 minutes, 5 minutes, or any time of aforementioned interval values.

In some embodiments, a step of reprogramming the human stem cells in the cell mixture includes transfecting a vector containing nucleic acids of nucleic reprogramming factors into the human stem cells in the cell mixture. In one embodiment, the nucleic reprogramming factors include one or more proteins selected from Oct family, Klf family, Sox family, Myc family, Lin family, or Glis family, such as Klf4, Oct3/4, Sox2, L-Myc, or Klf4. In one embodiment, CTS™ CytoTune™ Sendai vector (for example, CTS™ CytoTune™ 2.0 KOS containing genes of Klf4, Oct3/4, and Sox2, CTS™ CytoTune™ 2.0 hL-Myc containing genes of c-Myc, or CTS™ CytoTune™ 2.0 h Klf4 containing genes of Klf4) is transfected into human stem cells.

A universal human induced pluripotent stem cell is provided in some embodiments of the present disclosure, obtained from the foregoing method, in which universal hiPSCs include HLA class I gene and HLA class II gene, but showing no expression of HLA class I and HLA class II. In some embodiments, if universal hiPSCs and the mononuclear cells are mixed in a cell culture dish, and the initial concentration of the mononuclear cells is 1.5×10⁵ cells/mL, the survival ratio of universal hiPSCs is at least 95% after culture for two days.

In some embodiments of the present disclosure, a differentiated cell is provided, which can be obtained from differentiation of universal hiPSCs, in which the differentiated cell includes HLA class I gene and HLA class II gene, but showing no expression of HLA class I and HLA class II. In some embodiments, the differentiated cells include, but are not limited to, brain cells, neurons, astrocytes, glial cells, T cells, B cells, chondrocytes, bone cells, islet cells, adipose cells, heart cells, liver cells, kidney cells, lung cells, cardiomyocytes, skeletal muscle cells, eye cells, or osteogenic cells.

For further describing universal human induced pluripotent stem cells (universal hiPSCs) and a method of forming the same in some embodiments of the present disclosure, the following implementation is performed. It should be noted that the following embodiments are provided for exemplary purposes only, and do not limit the present invention.

### EXAMPLE 1: FORMATION OF UNIVERSAL HUMAN IPSCS

In the series of examples, two selection strategies are applied to form universal human iPSCs. Strategy 1 is to mix amniotic fluids from different subjects without genetic relationships with others, and strategy 2 is to mix amniotic fluids and mononuclear cells from different subjects without genetic relationships with others.

In detail, regarding strategy 1, first of all, the amniotic fluids from different pregnant women in the second trimester (13-28 weeks) were mixed with 0.5 mL and stored below 4° C. for more than two days, which includes the groups of mixing the amniotic fluids from two pregnant women (mix-2) and mixing the amniotic fluids from five pregnant women (mix-5). Furthermore, after the centrifugation of the mixed amniotic fluids at 350 xg for 5 minutes, the supernatant was discarded and the precipitate was obtained. The precipitate was cultivated in cell culture medium containing 60% MCDB 201 and 40% Dulbecco's modified Minimal Essential Medium (DMEM) in a CO₂ incubator at 37° C., and the selected AFSCs were cultivated until the confluence of the cells was about 78˜82%. Then, after 3 to 4 passages of the selected amniotic fluid stem cells, the cells were harvested. Finally, the genes of nucleic reprogramming factors were transfected into the selected amniotic fluid stem cells using CytoTune™-iPS 2.0 Sendai reprogramming kit (Invitrogen™), and the selected amniotic fluid stem cells were reprogrammed into universal human iPSCs, in which the universal human iPSCs formed by mixing amniotic fluids of two and five pregnant women were respectively referred to as hiPSC (mix-2) and hiPSC (mix-5). In addition, the control group (the human iPSCs directly formed by amniotic fluid without mixing with other amniotic fluids), referred to as hiPSC (single). The subsequent tests related to cell characteristics, differentiation ability and universal property (whether the allogeneic immune response was induced) were conducted by using hiPSC (mix-2) and hiPSC (mix-5).

It should be particularly emphasized that CytoTune™-iPS 2.0 Sendai reprogramming kit was performed by transfecting the vector containing the genes of nucleic reprogramming factors into the cells, and the vector existed in a plasmid form, without embedding into the chromosomes. Therefore, the genes of hiPSCs after being reprogrammed were at least 99.9% the same as the genes of the selected amniotic fluid stem cells before being reprogrammed. The risk of gene mutation was extremely low, and they were safe to use without side effects.

As for strategy 2, the method of obtaining amniotic fluids was the same as strategy 1. It should be noted that the amniotic fluid in strategy 2 was not treated with mixing with the amniotic fluids from different pregnant women as described in strategy 1; besides, the mononuclear cells were separated from the blood of the subject with no genetic relationship with the subject of the amniotic fluid. After obtaining the amniotic fluid and the mononuclear cells separately, the amniotic fluid and the mononuclear cells were mixed to contain 10⁴ mononuclear cells in 1 mL amniotic fluid. Next, the cell mixture was cultivated and selected in the same steps as strategy 1 to obtain the selected amniotic fluid stem cells, and the selected amniotic fluid stem cells were reprogrammed into universal human iPSCs, and cell characteristics, differentiation ability and universal property (whether the allogeneic immune response was induced) were conducted.

The principle of strategy 1 and strategy 2 was using allogeneic mononuclear cells with no genetic relationship with AFSCs to remove AFSCs with HLA class I and HLA class II, thereby increasing the performance ratio of AFSCs in the cell mixture that did not express HLA class I and HLA class II, and further, converting AFSCs that did not express HLA class I and HLA class II into universal hiPSCs at the step of reprogramming.

In order to simplify the context, the conditions and results of the relevant tests of hiPSC (mix-2) and hiPSC (mix-5) obtained by only strategy 1 about the cell characteristics, differentiation abilities and universal properties (whether the immune response was induced) were provided in the following description. It was emphasized that the universal human iPSCs obtained in strategy 2 and the hiPSC (mix-2) and hiPSC (mix-5) obtained in strategy 1 exhibited the correlated cell characteristics in the subsequent experimental conditions.

### EXAMPLE 2: CELL CHARACTERISTICS OF UNIVERSAL HUMAN IPSCS

HiPSC (mix-2) and hiPSC (mix-5) were cultivated in the cell culture dishes treated with vitronectin on the surface and containing Essential 8 cell culture medium, followed by performing cell characteristics analysis of hiPSC (mix-2) and hiPSC (mix-5) before and after differentiation.

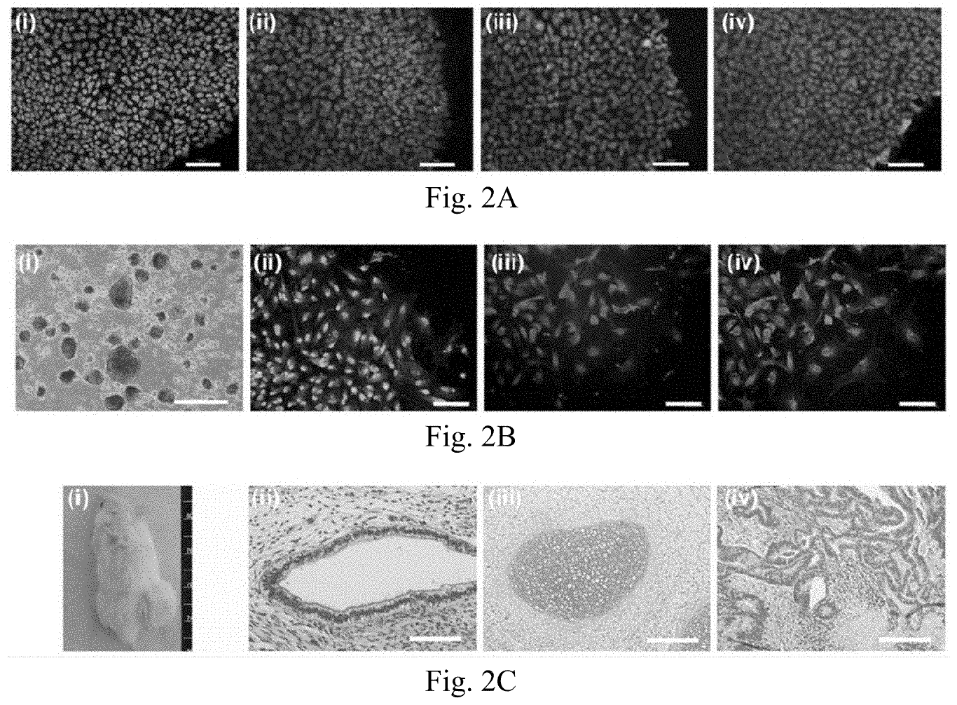

For detecting the cell pluripotency of pre-differentiated hiPSCs, immunostaining was performed on hiPSC (mix-2) and hiPSC (mix-5) cultivated for 20 passages to detect whether the pluripotent proteins (Oct4, Sox2, Nanog, and SSEA-4 were selected here) were expressed. In view of the correlated results between hiPSC (mix-2) and hiPSC (mix-5), the staining results of hiPSC (mix-5) were representatively presented, please refer to FIG. 2A.

According to FIG. 2A, the staining results represented that hiPSC (mix-5) expressed the pluripotent proteins continuously after 20 passages and retained the characteristics of pluripotent stem cells.

Furthermore, for testing whether hiPSC (mix-2) and hiPSC (mix-5) were differentiated into embryoid bodies, hiPSC (mix-2) and hiPSC (mix-5) were continuously cultivated for 21 passages, and then differentiated into embryoid bodies by conventional methods. Next, the expression of marker proteins in three embryonic layers (ectoderm, mesoderm, and endoderm) were detected by immunostaining, in which glial fibrillary acidic protein (GFAP) was the marker proteins for ectoderm, alpha-fetoprotein (AFP) was the marker protein for mesoderm, and smooth muscle actin (SMA) was marker protein in mesoderm. In view of the correlated results between hiPSC (mix-2) and hiPSC (mix-5), only the detected results of hiPSC (mix-5) were representatively presented, please refer to FIG. 2B.

FIG. 2B represented that embryoid bodies differentiated from hiPSC (mix-5) contained the three germ layer structure, and hiPSC (mix-5) had the differentiation ability to be normally differentiated into embryoid bodies.

Furthermore, for testing whether hiPSC (mix-5) and hiPSC (mix-2) could generate teratomas in vivo, 5×10⁶ hiPSC (mix-5) and hiPSC (mix-2) were subcutaneously injected into mice of NOD.CB17-Prkdc^{scid}/Jnarl model (NOD/SCID model). Eight weeks later, the teratomas formed in the mice body were processed by section staining to observe the internal structure. In view of the similar results between hiPSC (mix-2) and hiPSC (mix-5), the results of hiPSC (mix-5) were representatively presented; please refer to FIG. 2C.

(i) in FIG. 2C represented that teratoma was formed in mice injected with hiPSC (mix-5); (ii)-(iv) represented the tissue morphology of endoderm (ii), mesoderm (iii), and ectoderm (iv) in embryoid bodies were observed in the teratoma sections, indicating that hiPSC (mix-5) also had the differentiation ability into three germ layer cells in vivo.

### EXAMPLE 3: DIFFERENTIATION OF UNIVERSAL HIPSCS INTO CARDIOMYOCYTES

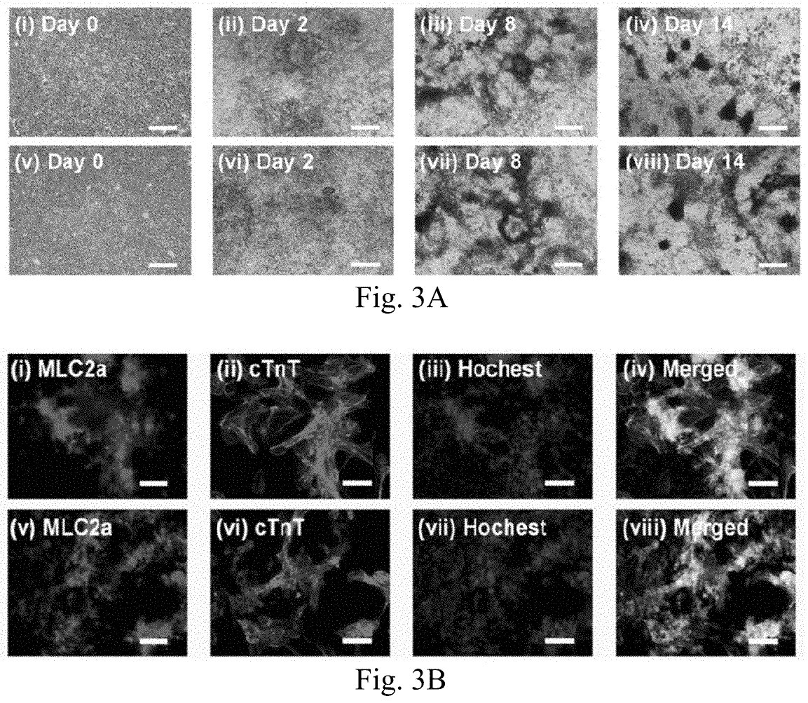

For observing the expression of HLA class I and HLA class II after the differentiation of universal human iPSCs into somatic cells, the differentiation method provided by Sharma et al. in 2015 (Sharma et al. Derivation of Highly Purified Cardiomyocytes from Human Induced Pluripotent Stem Cells Using Small Molecule-modulated Differentiation and Subsequent Glucose Starvation. J. Vis. Exp. (97), e52628, doi:10.3791/52628 (2015)) was slightly adjusted, and hiPSC (mix-2) and hiPSC (mix-5) were differentiated into cardiomyocytes by the differentiation method, including the steps of adding hiPSC (mix-2) and hiPSC (mix-5) to the cell culture dishes containing Essential 8 cell medium and treated with Matrigel on the surface until the cells were about 80-85% full. Next, the cell culture medium was replaced with Roswell Park Memorial Institute-1640 (RPMI-1640) cell culture medium, containing 2 wt % (weight percent concentration) of B27 without insulin and 6 μM CHIR99021 (GSK-3β inhibitor), and the cells were cultivated for two days. The cell culture medium was replaced with RPMI-1640 cell culture medium containing 2 wt % of B27 without insulin, and the cells were cultivated for two days. The cell culture medium was replaced with RPMI-1640 cell culture medium containing 5 μM IWR-1 (Wnt inhibitor) and 2 wt % of B27 without insulin, and the cells were cultivated for two days. Next, the cell culture medium was replaced with RPMI-1640 cell culture medium containing 2 wt % of B27 without insulin, and the cells were cultivated for one day. Finally, the cell culture medium was replaced with RPMI-1640 cell culture medium containing 2 wt % of B27, and the cells were continuously cultivated to obtain the differentiated cardiomyocytes. The morphology of the cells at each time point was photographed with the microscope, and the results were summarized into FIG. 3A, in which (i)-(iv) were the differentiation process of hiPSC (mix-2), and (v)-(viii) were the differentiation process of hiPSC (mix-5), and it was observed that the morphology of the cells was gradually transformed into cardiomyocytes.

Furthermore, the immunostaining method was used to detect whether the specific proteins of cardiomyocytes differentiated from hiPSC (mix-2) and hiPSC (mix-5) (MLC2a and cTnT were selected here) were expressed.

Please refer to FIG. 3B, in which (i)-(iv) were the cardiomyocytes differentiated from hiPSC (mix-2), and (v)-(viii) were the cardiomyocytes differentiated from hiPSC (mix-5), and whether the cardiomyocytes differentiated from hiPSC (mix-2) or hiPSC (mix-5) normally expressed specific proteins of cardiomyocytes, indicating that hiPSC (mix-2) or hiPSC (mix-5) could be differentiated into somatic cells normally.

### EXAMPLE 4: EXPRESSION OF HLA CLASS I AND HLA CLASS II IN CELLS BEFORE AND AFTER DIFFERENTIATION

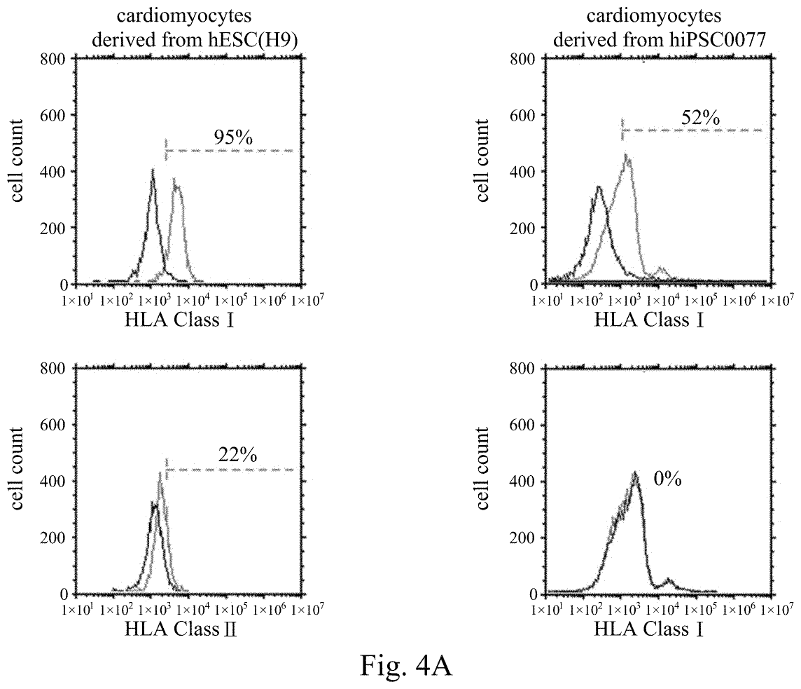

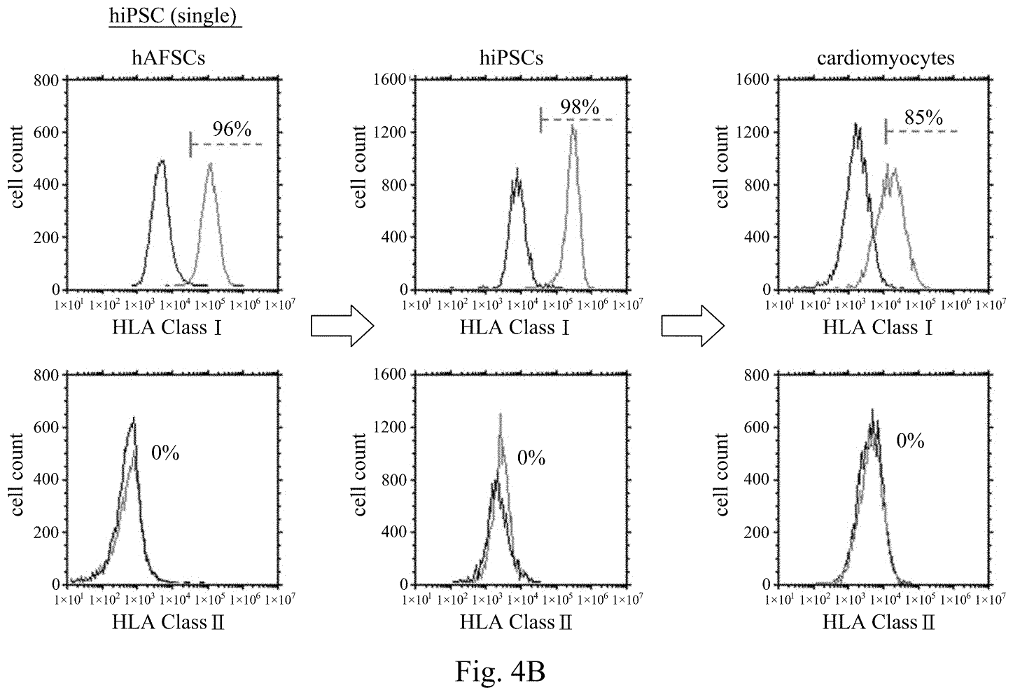

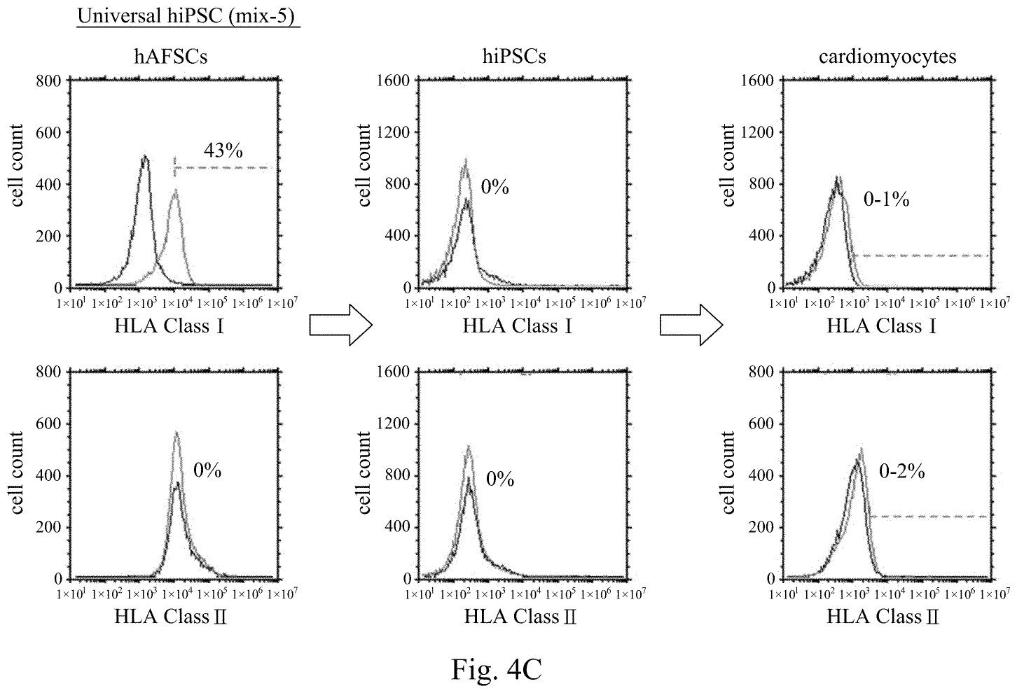

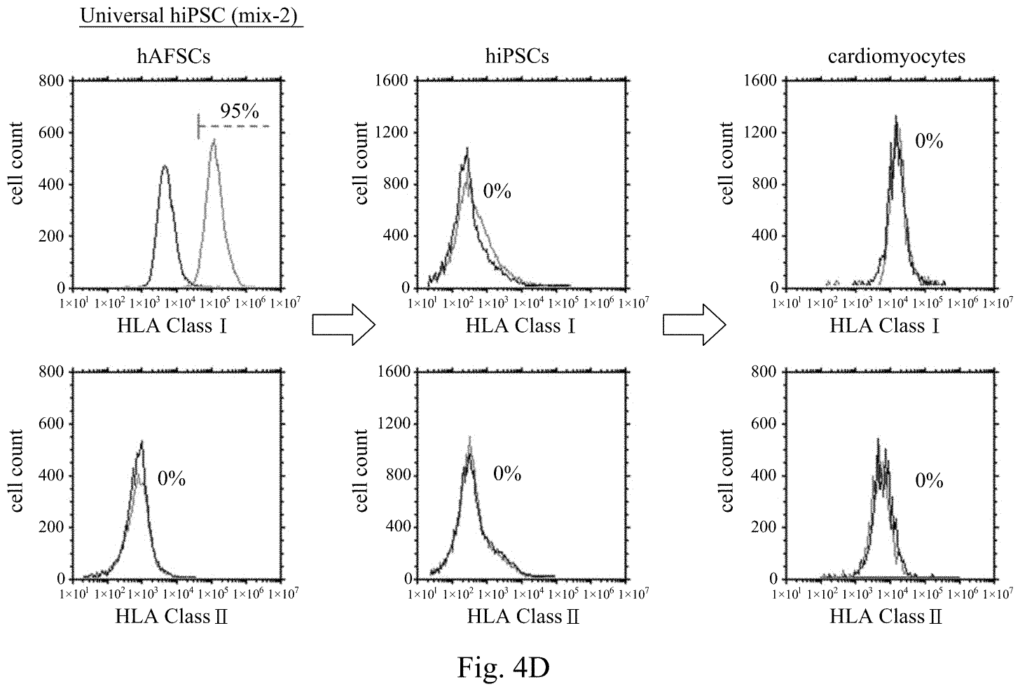

For observing the expression of HLA class I and HLA class II before and after cell differentiation, HLA class I and HLA class II in hiPSC (mix-2), hiPSC (mix-5), and the cardiomyocytes differentiated from hiPSC (mix-2) and hiPSC (mix-5) were targeted using HLA class I antibody and HLA class II antibody, in which hiPSC (mix-2) and hiPSC (mix-5) were cultivated for at least 15 passages. Furthermore, the expressions of HLA class I and HLA class II in each cell group was analyzed using the flow cytometer (BD Accuri™ C6, BD Biosciences, Flanklin Lakes, N.J., USA). At the same time, the expression of HLA class I and HLA class II in the cardiomyocytes differentiated from current human embryonic stem cells (hESCs) (H9, WiCell Research Institute, Inc., Madison, Wis., USA) (referred to as hESC (H9)), the cardiomyocytes differentiated from hiPSCs (HPS0077, Riken BioResource Center, Tsukuba, Japan) (referred to as hiPSC0077) and hiPSC (single), served as a control group, formed by amniotic fluid and not treated with other amniotic fluids or mononuclear cells, was also analyzed by the flow cytometry, and the results were summarized into FIGS. 4A to 4D. FIGS. 4A and 4B were the analysis results of the control group, and FIGS. 4C and 4D were the analysis results of hiPSC (mix-2) and hiPSC (mix-5).

FIGS. 4A to 4B represented that HLA class I or HLA class II were expressed in the cardiomyocytes differentiated (or derived) from current hESC (H9), the cardiomyocytes differentiated from hiPSC0077, hiPSC (single), without mixing with amniotic fluid or being treated with mononuclear cells, or the cardiomyocytes differentiated from hiPSC (single). FIGS. 4C to 4D represented that no HLA class I or HLA class II was expressed in hiPSC (mix-2) and hiPSC (mix-5), formed in Example 1 and cultivated for at least 15 passages, and in the cardiomyocytes differentiated from hiPSC (mix-2) and hiPSC (mix-5), confirming hiPSC (mix-2) and hiPSC (mix-5) were universal hiPSCs described in this present disclosure.

### EXAMPLE 5: NO IMMUNE RESPONSE INDUCED BY ALLOGENEIC MONONUCLEAR CELLS IN UNIVERSAL HIPSCS

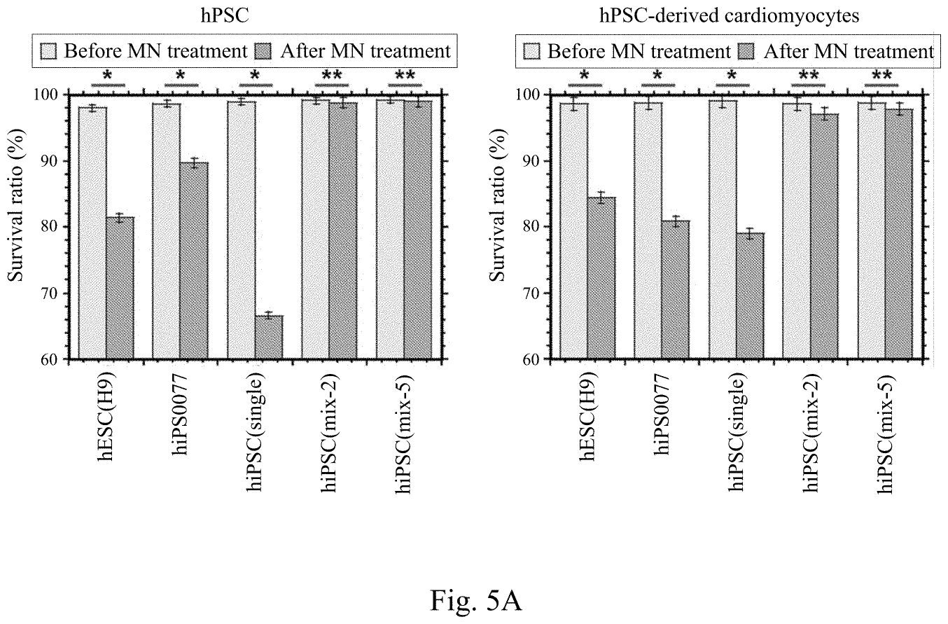

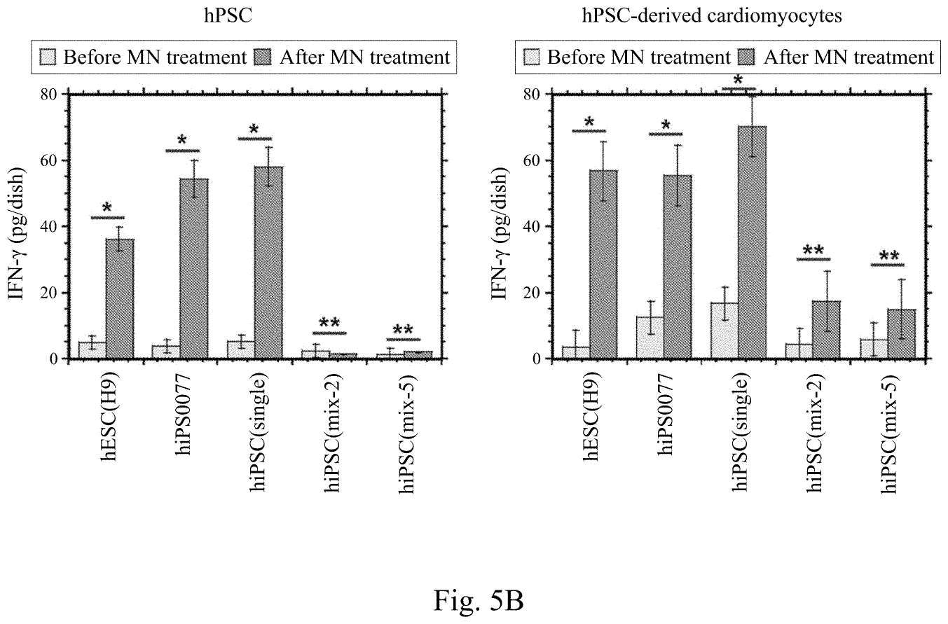

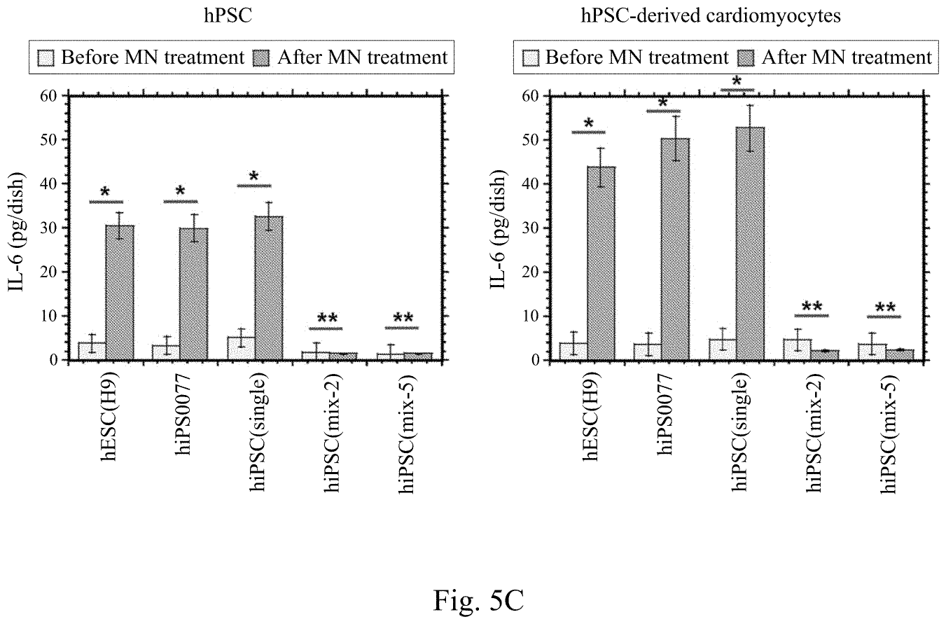

For further confirming whether the immune response was induced while hiPSC (mix-2) and hiPSC (mix-5) were transplanted into other subjects, the mononuclear cells separated from another subjects with no genetic relationship with the other cells were added to the cell culture dishes respectively cultivated with hESC (H9), hiPSC0077, hiPSC (single), hiPSC (mix-2), hiPSC (mix-5) and the cardiomyocytes differentiated from the aforementioned cells, and the concentration of mononuclear cells was 1.5×10⁵ cells/mL. After the cells had been cultivated continuously for two days, the cell survival ratio and the concentration of cytokines (IFN-γ and IL-6 were selected here) were analyzed, in which the cell survival ratio was acquired by adding 7-amino-actinomycin D (7-AAD) to distinguish between the live cells and the dead cells and then analyzing with the flow cytometer. Four replicate data was conducted in each group, and the correlation of the data was analyzed by unpaired Student's-tests. If the P value was less than 0.05 (indicated with single star), it was considered statistically significant, and if the P-value was greater than 0.05 (indicated with two stars), it was considered statistically insignificant. For the results of cell survival ratio, please refer to FIG. 5A. For the concentration of cytokine IFN-γ, please refer to FIG. 5B. For the concentration of cytokine IL-6, please refer to FIG. 5C.

FIG. 5A represented that the survival ratio of the groups of hiPSC (mix-2), hiPSC (mix-5), and the cardiomyocytes differentiated from hiPSC (mix-2) and hiPSC (mix-5) were maintained at a level higher than 95% after the treatment of mononuclear cells; however, as for the control group, the survival ratio of hESC (H9), hiPSC0077, hiPSC (single) and the cardiomyocytes differentiated from the these cell groups was significantly decreased, in which hiPSC0077, the group with the highest survival ratio, was not higher than 90%.

FIGS. 5B and 5C represented that the changes of cytokines IFN-γ and IL-6 in the cardiomyocyte groups differentiated from hiPSC (mix-2) and hiPSC (mix-5) after the treatment of mononuclear cells had no significant difference from which before the treatment of mononuclear cells. However, a large amount of IFN-γ and IL-6 in hESC (H9), hiPSC0077, hiPSC (single) and the cardiomyocyte groups differentiated from these cells after the treatment of mononuclear cells was detected and had statistically significant differences from which before the treatment of mononuclear cells.

That is, no immune response was induced by the differentiated cells from hiPSC (mix-2) and hiPSC (mix-5) provided in Example 1 during allogeneic transplantation, and the differentiated cells had high safety, compared with the conventional hESC and hiPSC, with potential problem of immune response induction during allogeneic transplantation.

### EXAMPLE 6: OBTAINING UNIVERSAL HIPSCS FROM OTHER SOMATIC CELLS

In addition to the method disclosed in the abovementioned Example 1, the method of obtaining universal hiPSCs from amniotic fluid, strategy 2 of Example 2 was also referred, in which universal hiPSCs were obtained by treating somatic cells, such as adipose cells or the tissues of dental pulp, umbilical artery, umbilical cord blood, adipose, marrow, or the like, with mononuclear cells at the concentration of 1.5×10⁵ cells/mL and reprogramming the selected stem cells into hiPSCs.

Universal hiPSCs and a method of forming the same are provided in some embodiments of the present disclosure, including mixing a cell group containing the stem cells with mononuclear cells with different HLA class I and HLA class II from the stem cells (e.g., from other subjects with no genetic relationship with the source subjects of the stem cells), selecting the stem cells by mononuclear cells, and then reprogramming the selected stem cells, forming universal hiPSCs that do not express HLA class I and HLA class II and without chromosome change.

The method of forming universal hiPSCs provided in some embodiments of the present disclosure at least has advantages including: 1. easy operation; 2. wide application scope, which can be applied to any somatic cell containing stem cells, or the like. In addition, universal hiPSCs obtained in some embodiments of the present disclosure at least have advantages including: 1. highly safe and not directed to the editing of chromosomes; 2. high universality, not expressing HLA class I and HLA class II, suitable in allogeneic transplantation, and not inducing immune response, or the like. Therefore, universal hiPSCs and the forming method provided in some embodiments of the present disclosure have high application value for clinical uses.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A method of forming universal human induced pluripotent stem cells, comprising steps of:
providing a first cell group including human stem cells;
providing a second cell group including human mononuclear cells, wherein an expression form of human leukocyte antigen-1 (HLA class I) or an expression form of human leukocyte antigen-2 (HLA class II) of the human mononuclear cells are different from an expression form of HLA class I or an expression form of HLA class II of the first cell group;
mixing the first cell group and the second cell group to form a cell mixture;
maintaining the cell mixture below 30° C. for at least one day; and
reprogramming the human stem cells in the cell mixture to obtain universal human induced pluripotent stem cells, wherein the universal human induced pluripotent stem cells include HLA class I gene and HLA class II gene, but no HLA class I and HLA class II expressions.

2. The method of claim 1, wherein at the step of providing the first cell group including the human stem cells, the first cell group is obtained from first amniotic fluid, dental pulp, umbilical cord, umbilical cord blood, adipose, marrow, or combination thereof.

3. The method of claim 1, wherein at the step of providing the second cell group including the human mononuclear cells, the second cell group is obtained from spinal fluid, second amniotic fluid, blood, or combination thereof.

4. The method of claim 1, wherein
at the step of providing the first cell group including the human stem cells is provided, the first cell group is obtained from first amniotic fluid; and
at the step of providing the second cell group including the human mononuclear cells, the second cell group is obtained from second amniotic fluid, wherein the first amniotic fluid and the second amniotic fluid are obtained from different subjects.

5. The method of claim 4, wherein the first amniotic fluid and the second amniotic fluid are at least 0.5 mL, respectively.

6. The method of claim 4, wherein at the step of providing the second cell group including the human mononuclear cells, the second amniotic fluid includes a plurality of amniotic fluids derived from different subjects, wherein the plurality of amniotic fluids include a plurality of human mononuclear cells.

7. The method of claim 1, wherein
at the step of providing the first cell group including human stem cells, the first cell group is obtained from first amniotic fluid; and
at the step of providing the second cell group including the human mononuclear cells, the second cell group is human mononuclear cells.

8. The method of claim 7, wherein the step of mixing the first cell group and the second cell group includes mixing the first amniotic fluid and a buffer including the human mononuclear cells to form the cell mixture, wherein the first amniotic fluid is at least 0.5 mL, and a concentration of the human mononuclear cells in a cell culture dish containing the cell mixture is 1.5×10⁴ to 1.5×10⁶ cells/mL.

9. The method of claim 1, wherein the step of reprogramming the human stem cells in the cell mixture includes transfecting a vector containing nucleic acids of nucleic reprogramming factors into the human stem cells in the cell mixture.

10. A universal human induced pluripotent stem cell, comprising HLA class I gene and HLA class II gene, but no HLA class I and HLA class II expressions.

11. A differentiated cell, comprising HLA class I gene and HLA class II gene, but no HLA class I and HLA class II expressions.
